(19) United States
(12) Patent Application Publication
(10) Pub. No.: US 2026/0232178 A1
(43) Pub. Date: Aug. 13, 2026

(54) MEDICAL DEVICES HAVING A REDUCED PROFILE AND RELATED METHODS OF USE

(71) Applicant: Boston Scientific Medical Device Limited, Galway (IE)

(72) Inventors: Yao CHEN, Shanghai (CN); Linshanzi PAN, Shanghai (CN); Shibo WANG, Shanghai (CN); Mengxiang LUO, Shanghai (CN); Cheng ZHANG, Shanghai (CN)

(73) Assignee: Boston Scientific Medical Device Limited, Galway (IE)

(21) Appl. No.: 19/532,113

(22) Filed: Feb. 6, 2026

(30) Foreign Application Priority Data
Feb. 7, 2025 (CN) 2025101359359

(51) Int. Cl. A61B 1/05; A61B 1/00; A61B 1/005; A61B 1/018; A61B 1/06

(52) U.S. Cl. CPC A61B 1/05; A61B 1/00071; A61B 1/00117; A61B 1/00119; A61B 1/0052; A61B 1/018; A61B 1/0676; A61B 2560/0462

(57) ABSTRACT

Medical devices are described, including a medical device having a shaft. The shaft may include a working channel extending from a proximal end of the shaft to an opening at a distal end of the shaft, and a plurality of optical fibers surrounding the working channel. The shaft also may include an imaging device at the distal end of the shaft.

### CROSS-REFERENCE TO RELATED APPLICATIONS

This application claims the benefit of priority to Chinese Application No. 2025101359359, filed on February 7, 2025, which is incorporated by reference herein in its entirety.

### TECHNICAL FIELD

Various aspects of this disclosure relate generally to medical devices and related methods of use. In particular, aspects of this disclosure relate to medical devices having a reduced profile.

### BACKGROUND

During ureteroscopic procedures, a ureteroscope or other type of scope may be introduced into a subject's body, e.g., introduced into the urinary tract of the subject to locate, remove, and/or fragment kidney stones. A challenge associated with ureteroscopic procedures is management of intrarenal pressure. Under normal physiological conditions, intrarenal pressure typically remains below 10 mmHg. However, during ureteroscopic procedures, intrarenal pressure may rise rapidly due to aspects of the medical procedure, e.g., continuous irrigation. High intrarenal pressure may be associated with an increased risk of post-treatment complications such as pyelovenous backflow and subsequent sepsis, and kidney damage.

### SUMMARY

Each of the aspects disclosed herein may include one or more aspects of the features described in connection with any of the other disclosed aspects.

This disclosure includes a medical device having a shaft, wherein the shaft comprises a working channel extending from a proximal end of the shaft to an opening at a distal end of the shaft, and a plurality of optical fibers surrounding the working channel. The shaft also may comprise an imaging device at the distal end of the shaft.

According to some aspects, the plurality of optical fibers may completely surround the working channel and may be arranged in a ring. In some examples, the plurality of optical fibers may define walls of the working channel. In some examples, the working channel may have a cross-sectional dimension, e.g., a diameter, of approximately 3.4 French to approximately 3.8 French, e.g., about 3.6 French. Optionally, the plurality of optical fibers may surround the imaging device. Additionally or alternatively, the plurality of optical fibers may be encapsulated by a material and/or may comprise a coating. In some examples, the plurality of optical fibers may include epoxy resin adhesive or a UV-curable adhesive. In some examples, each optical fiber of the plurality of optical fibers may have an outer diameter of approximately 250 μm or less, e.g., about 100 μm to about 200 μm. Optionally, the imaging device may include a CMOS sensor. Additionally or alternatively, the shaft may further include at least one irrigation channel and/or suction channel. According to some aspects, the shaft may have a non-circular cross-sectional shape. In some examples, the shaft may have an outer diameter less than approximately 9 French. In some examples, the medical device may further include a handle coupled to the shaft and the handle may include a port in communication with the working channel.

This disclosure also includes, for example, a medical device having a handle and a shaft extending distally from the handle, wherein the shaft comprises a working channel extending from a proximal end of the shaft to an opening at a distal end of the shaft, and a plurality of optical fibers. The shaft also may comprise an imaging device at the distal end of the shaft. The plurality of optical fibers may define walls of the working channel. In some examples, an outer diameter of each optical fiber of the plurality of optical fibers may be approximately 250 μm or less, e.g., about 50 μm to about 250 μm, or about 100 μm to about 200 μm. In some examples, the plurality of optical fibers may be arranged in a ring.

This disclosure also includes, for example, a medical device with a shaft that comprises a working channel extending from a proximal end of the shaft to an opening at a distal end of the shaft, wherein at least a portion of the shaft adjacent to the working channel from the proximal end of the shaft to the distal end of the shaft includes an elastic material. The working channel may be configured to expand by stretching of the elastic material. The medical device also may comprise an imaging device and a light source at the distal end of the shaft. Optionally, the shaft may have a non-uniform outer diameter when the working channel is in a contracted state and when the working channel is in an expanded state.

### BRIEF DESCRIPTION OF FIGURES

The accompanying drawings, which are incorporated in and constitute a part of this application, illustrate exemplary aspects of this disclosure and together with the description, serve to explain the principles of this disclosure.

FIGS. 1A and 1B illustrate an exemplary medical device, in accordance with some aspects of this disclosure.

FIG. 2A illustrates a distal end portion of an exemplary medical device, in accordance with some aspects of this disclosure.

FIG. 2B illustrates a distal portion of an exemplary working channel of the medical device of FIG. 2A, in accordance with some aspects of this disclosure.

FIG. 2C illustrates a distal portion of another exemplary working channel of a medical device such as the medical device, in accordance with some aspects of this disclosure.

FIG. 3 illustrates a distal end portion of another exemplary medical device, in accordance with some aspects of this disclosure.

FIG. 4A illustrates a cross-sectional view of a distal end portion of another exemplary medical device in a first configuration, in accordance with some aspects of this disclosure.

FIG. 4B illustrates a distalmost face of the distal end portion of the medical device of FIG. 4A in the first configuration, in accordance with some aspects of this disclosure.

FIG. 4C illustrates a cross-sectional view of the distal end portion of the medical device of FIG. 4A in a second configuration, in accordance with some aspects of this disclosure.

FIG. 4D illustrates the distalmost face of the distal end portion of the medical device of FIG. 4A in the second configuration, in accordance with some aspects of this disclosure.

### DETAILED DESCRIPTION

Particular aspects of the disclosure are described in greater detail below. The terms and definitions provided herein control, if in conflict with terms and/or definitions incorporated by reference. Wherever possible, the same or similar reference numbers will be used through the drawings to refer to the same or like parts.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although ureteroscopes are referenced herein for illustration purposes, it will be appreciated that the disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject’s body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, laparoscopes, cystoscopes, aspiration scopes, sheaths, catheters, or any other suitable delivery device or medical device may be used in connection with the features described herein.

This disclosure includes medical devices having a reduced profile, while maintaining and/or enhancing functionality of various components (e.g., imaging devices, light sources, working channels, articulation or other wires or cables, articulation links, etc.) of the medical device. The reduced profile of the medical devices herein may assist with maintaining suitable intrarenal pressure of the patient, navigating through tortuous body anatomy, and/or reducing patient discomfort during a medical procedure, among other aspects. The medical devices herein may comprise a handle and a shaft including a working channel extending therethrough. The outer diameter of the shaft may have a reduced cross-sectional size, while maintaining or providing a working channel having a size large enough to allow for passage of a medical instrument through the working channel. For example, an outer diameter of the shafts herein may be less than approximately 9 French, e.g., from about 4 French to about 9 French.

According to some aspects of the disclosure, the shaft may include imaging components surrounding and/or arranged around the working channel. For example, the shaft may include an imaging device (e.g., a CMOS sensor) and a plurality of light sources, such as a plurality of optical fibers. The plurality of optical fibers may surround the working channel and/or may define walls of the working channel. For example, the plurality of optical fibers may be relatively small in diameter, e.g., taking up less space within the shaft. The plurality of optical fibers may provide enhanced lighting (e.g., increased luminance) at the distal end of the shaft, e.g., compared to one optical fiber or one other light source.

According to some aspects of the disclosure, the shaft may additionally or alternatively include an at least partially expandable working channel, e.g., provided by an elastic material of the shaft. The working channel may be configured to transition between a contracted state (e.g., a relaxed state) and an expanded state. The working channel may be biased to the contracted state to maintain a reduced profile of the shaft. For example, the outer diameter of the shaft may be temporarily enlarged only when needed, e.g., when a medical instrument having a size larger than the working channel in the contracted state is extended through the working channel, thereby causing the working channel to temporarily expand.

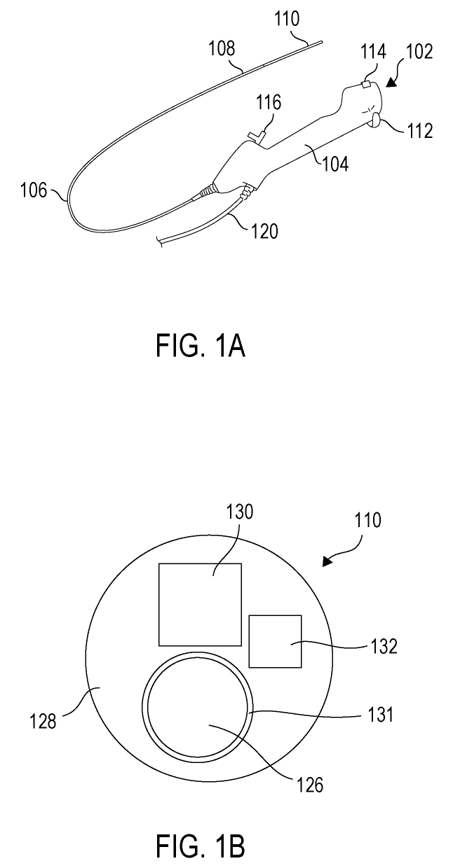

An exemplary medical device 102 will now described with reference to FIGS. 1A and 1B. Medical device 102 (e.g., a ureteroscope) may include a handle 104 and a shaft 106 extending distally from handle 104. Shaft 106 may be sufficiently flexible to facilitate navigation of shaft 106 through tortuous anatomical passages in a subject's body. In some aspects, shaft 106 may have a substantially circular cross-sectional shape. Shaft 106 may include a steerable section 108 and a distal tip 110. In some examples, distal tip 110 may be integral with a remainder of shaft 106. In other examples, distal tip 110 may be a separate piece of shaft 106 that is coupled to a distal portion of shaft 106. For example, distal tip 110 may comprise an end cap fixedly or detachably coupled to a distal end of shaft 106.

Handle 104 may include one or more actuators for controlling aspects of medical device 102, e.g., via user input. The one or more actuators may include, for example, a lever, switch, button, knob, and/or any other suitable type of mechanism for receiving and transmitting user input. For example, handle 104 may include a first actuator 112 (e.g., a lever) and a second actuator 114 (e.g., a button). First actuator 112 may be configured to articulate steerable section 108 and distal tip 110 in one or more directions, e.g., via one or more articulation cables coupled to articulation links within steerable section 108. Second actuator 114 may be configured to actuate and/or control other aspects of medical device 102, e.g., turning on/off light sources (e.g., optical fiber(s)) and/or controlling an imaging device to capture images. Handle 104 may include a port 116 in communication with a working channel 126 of shaft 106. A medical instrument, e.g., a laser fiber, grasper, retrieval device, etc., may be inserted through port 116 and moved distally through working channel 126 of shaft 106.

Handle 104 may be coupled to an umbilicus 120. Umbilicus 120 may extend from handle 104 and may include or carry wires, cables, and/or conduits configured to provide, e.g., power, signals, or fluids to and/or from handle 104. For example, umbilicus 120 may connect handle 104 to one or more user interfaces, monitors, control units, displays, etc.

As shown in FIG. 1B, medical device 102 may include imaging components such as an imaging device 130 (e.g., a camera or other imager, etc.) and one or more light sources 132 (e.g., LEDs, optical fibers, etc.) at distal tip 110. Working channel 126 may extend from a proximal end of shaft to an opening 131 of a distalmost face 128 of distal tip 110.

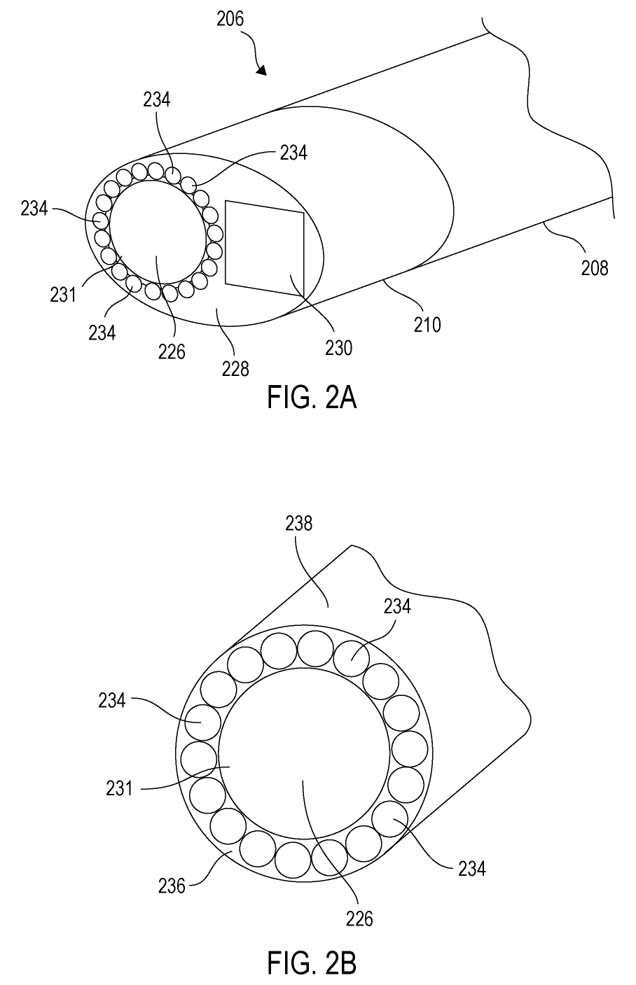

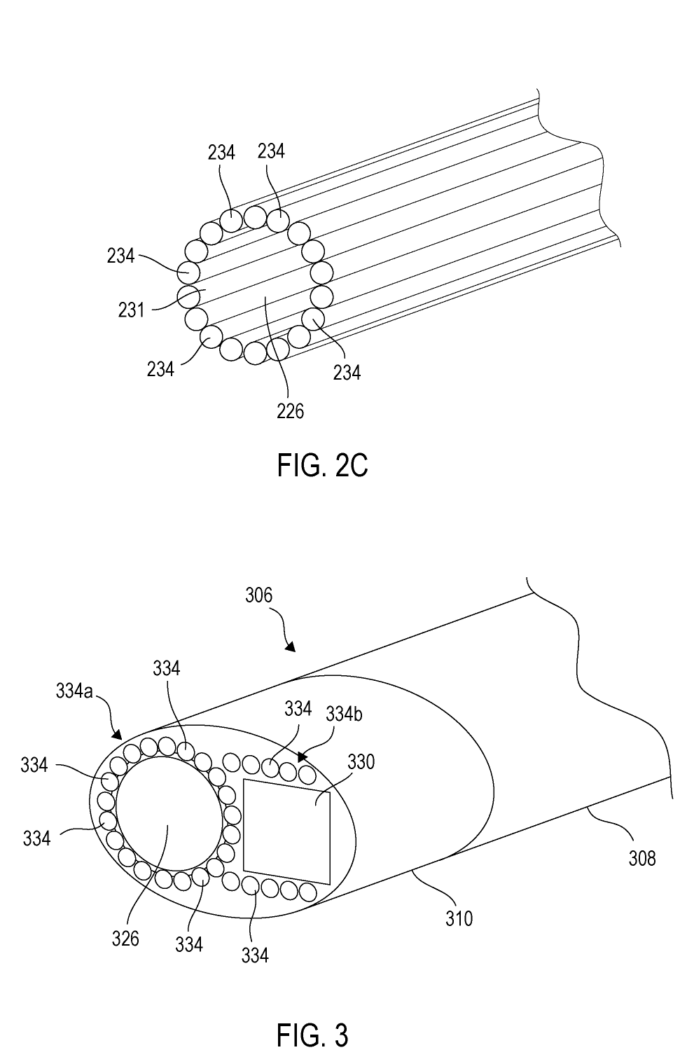

FIG. 2A illustrates a distal end portion of an exemplary shaft 206 of another medical device. Shaft 206 may be coupled to a handle such as handle 104 of medical device 102 (e.g., in place of shaft 106) and may include any of the features of shaft 106 unless otherwise specified. Shaft 206 may include a steerable section 208, a working channel 226, and a distal tip 210 with an imaging device 230.

In this example, shaft 206 may include a plurality of optical fibers 234 as light sources surrounding working channel 226. For example, a single layer of optical fibers 234 may substantially or completely surround a circumference of working channel 226. The plurality of optical fibers 234 may be relatively small in diameter. For example, each optical fiber of the plurality of optical fibers 234 may have an outer diameter of approximately 250 μm or less, e.g., an outer diameter ranging from about 50 μm to about 250 μm, or from about 100 μm to about 200 μm. Imaging device 230 may comprise a CMOS sensor. For example, the CMOS sensor may have dimensions of approximately 1.0 mm x 1.0 mm or less.

Working channel 226 may extend from a proximal end of shaft 206 to an opening 231 of a distalmost face 228 of distal tip 210. Each optical fiber of the plurality of optical fibers 234 may extend longitudinally through shaft 206 and adjacent to working channel 226. Each optical fiber of the plurality of optical fibers 234 may terminate at or proximate distalmost face 228 of distal tip 210. As shown in FIGS. 2A and 2B, the plurality of optical fibers 234 may completely surround a circumference of working channel 226. For example, the plurality of optical fibers 234 may be arranged in a ring completely surrounding a circumference of working channel 226. FIG. 2C shows a variation of the plurality of optical fibers 234 as discussed below.

In some aspects, one or more optical fibers 234 may be separated by an open channel, e.g., channels that terminate at distalmost face 228, useful for irrigation and/or suction. For example, referring to the configuration shown in FIG. 2B, one optical fiber 234 as depicted may be replaced with a tube (e.g., a capillary tube) useful for irrigation and/or suction. In such cases, the ring-like arrangement of the plurality of optical fibers 234 may include one, two, or more open channels between optical fibers 234.

Shaft 206 may have a circular or non-circular (e.g., an elliptical) cross-sectional shape. Shaft 206 may have a relatively compact design as compared to other medical devices, while maintaining a cross-sectional dimension of working channel 226 large enough to allow passage of a medical instrument therethrough. For example, a diameter of working channel 226 may range from about 3 French to about 4 French, such as approximately 3.4 French, 3.6 French, or 3.8 French. The plurality of optical fibers 234 may define walls of, or otherwise may be used to form, working channel 226. In some examples, working channel 226 may be defined by or completely surrounded by optical fibers 234. In at least one example, at least 15 optical fibers 234, e.g., from 20 to 25 optical fibers, may surround working channel 226 having a diameter of about 3.4 French to about 3.8 French. In at least one example, 20-22 optical fibers 234, e.g., 21 optical fibers 234, may completely surround (e.g., define the walls of) working channel 226 when working channel 226 has a diameter of approximately 3.6 French.

The plurality of optical fibers 234 may be fixed in place and/or fixed to one another to form working channel 226 using different methods, which will now be discussed with reference to FIGS. 2B and 2C.

In some aspects, the plurality of optical fibers 234 may be fixed in place to form working channel 226 using a reflow process. The plurality of optical fibers 234 may be encapsulated by or may be surrounded by a material 236 by reflowing. Material 236 may comprise a polymer, for example. Exemplary polymers suitable for material 236 include, but are not limited to, polyether block amide. For example, a mandrel may be inserted into a catheter (e.g., a tube) comprising material 236. The plurality of optical fibers 234 may then be placed around an outer surface of the catheter in the configuration illustrated in FIG. 2B. The plurality of optical fibers 234 may be surrounded by a heat-shrinkable tube or cover having a melting point greater than a melting point of material 236. In some examples, the heat-shrinkable tube or cover may comprise a polymer, such as, e.g., fluorinated ethylene propylene. Heat may then be applied. As the temperature increases, the heat-shrinkable cover may shrink around the plurality of optical fibers 234. Once the temperature reaches the melting point of material 236, material 236 may melt and flow to surround the plurality of optical fibers 234. Heat may be removed and material 236 may be solidified by cooling. The mandrel may then be removed leaving a tubular structure including working channel 226 surrounded by the plurality of optical fibers 234. Material 236 together with the plurality of optical fibers 234 may define a wall 238 of working channel 226.

As shown in the variation of FIG. 2C, the plurality of optical fibers 234 may be fixed to one another to form walls of working channel 226 without material 236 and without using a reflow process. For example, medical-grade adhesives, such as epoxy resin or UV-curable adhesives, may be used to fix the plurality of optical fibers 234 to one another.

Other methods may be utilized to form working channel 226 using the plurality of optical fibers 234. In some examples, a multi-lumen tube may be formed, e.g., via an extrusion process. The multi-lumen tube may include a central lumen (e.g., working channel 226) and a plurality of lumens for receiving the plurality of optical fibers 234 arranged around the central lumen. Each of the plurality of optical fibers 234 may be secured within their respective lumens of the multi-lumen tube, e.g., via heat fusion processes.

In other examples, 3D printing techniques, such as selective laser sintering, may be utilized to form high-precision plastic tubes arranged in a desired configuration (e.g., arranged in a ring-like configuration) to form working channel 226. Each tube may include a lumen for receiving a respective optical fiber 234.

In some examples, film wrapping techniques may be utilized. For example, the plurality of optical fibers 234 may first be arranged in a desired configuration (e.g., arranged side by side). The plurality of optical fibers 234 may then be wrapped with a film to fix the plurality of optical fibers 234 in place. The plurality of optical fibers 234 may then be rolled into a required size and shape to form working channel 226. For example, the plurality of optical fibers 234 may be rolled into a circular or tubular structure to form working channel 226.

Optionally, shaft 206 may include additional features to protect the plurality of optical fibers 234 from the external environment. For example, shaft 206 may be a multi-lumen extruded shaft including designated lumens for receiving the plurality of optical fibers 234. In some examples, the plurality of optical fibers 234 may be twisted or may be braided. Additionally or alternatively, the plurality of optical fibers 234 may include a reinforced inner liner and/or outer liner (e.g., a coating). The inner liner and/or outer liner may comprise a suitable polymer such as, e.g., polyamide, polyurethane, polyethylene, and/or polytetrafluoroethylene. For example, the reinforced inner liner and/or outer liner may be co-molded onto the plurality of optical fibers 234.

FIG. 3 illustrates a distal end portion of another exemplary shaft 306 of a medical device according to aspects of this disclosure. Shaft 306 may be similar to shaft 206 but include additional optical fibers surrounding an imaging device to, e.g., provide enhanced lighting at a distal end of shaft 306. Shaft 306 may include a steerable section 308, a distal tip 310 with an imaging device 330, a working channel 326, and a plurality of optical fibers 334. The plurality of optical fibers 334 may include a first set 334a of optical fibers 334 and a second set 334b of optical fibers 334, with first set 334a of optical fibers 334 being similar to the plurality of optical fibers 234 in that first set 334a of optical fibers 334 may completely surround a circumference of working channel 326. Second set 334b of optical fibers 334 may partially surround imaging device 330.

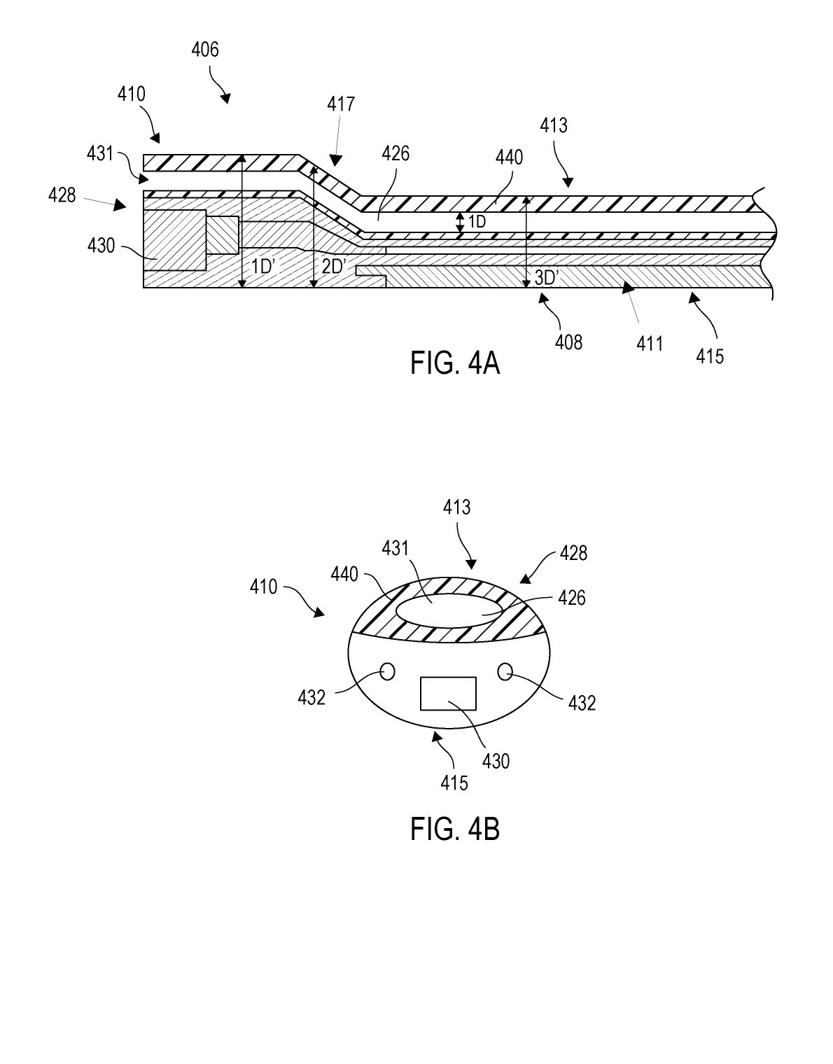

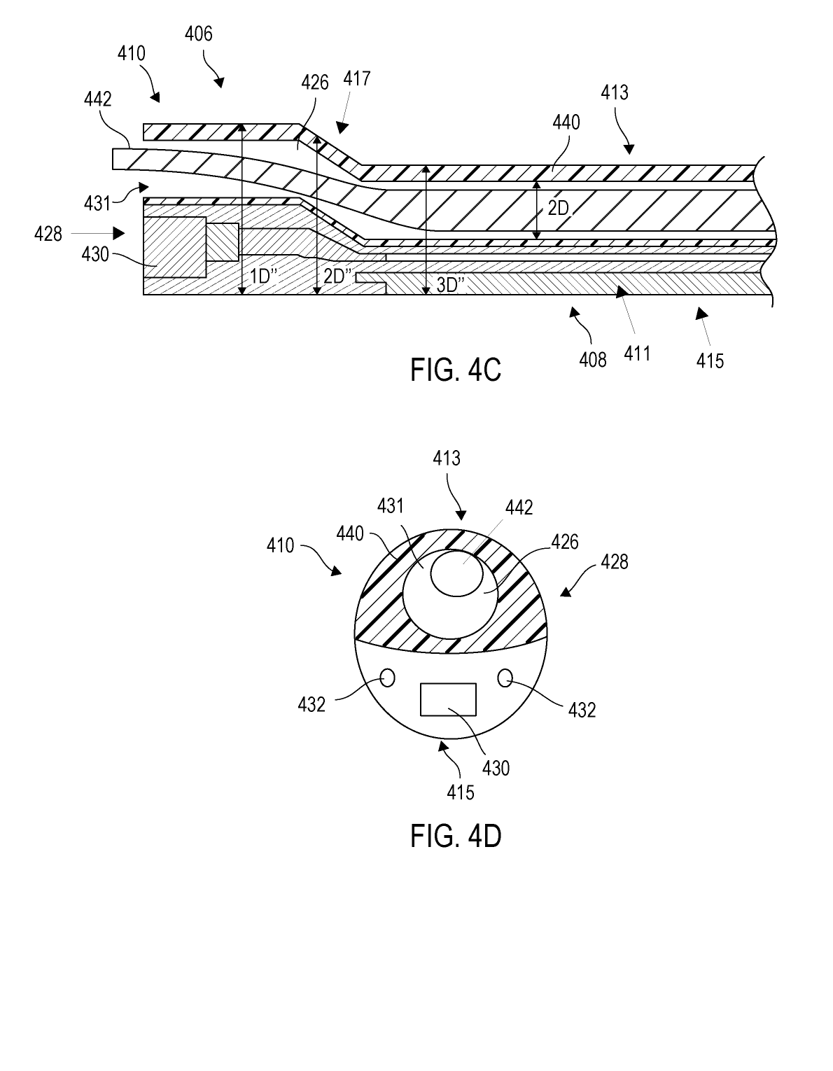

FIGS. 4A-4D illustrate features of a distal end portion of another exemplary shaft 406 of a medical device having an expandable working channel 426. Shaft 406 may be used in combination with handle 104 of medical device 102 (e.g., in place of shafts 106, 206, 306). Shaft 406 may include a steerable section 408 and a distal tip 410.

FIG. 4A illustrates a cross-sectional view of the distal end portion of shaft 406 and FIG. 4B illustrates a distalmost end of shaft 406, in a contracted state. FIG. 4C illustrates a cross-sectional view of the distal end portion of shaft 406 and FIG. 4D illustrates the distalmost end of shaft 406, in an expanded state.

Shaft 406 may include an elastic portion 413 and a remaining portion 415. Each portion 413, 415 may extend from a proximal end of shaft 406 to the distalmost end of shaft 406 (i.e., a distalmost face 428 of distal tip 410). For example, portions 413 and 415 may be on opposite sides of shaft 406 from the proximal end of shaft 206 to the distalmost end of shaft 406. Working channel 426 may extend alongside elastic portion 413 from the proximal end of shaft 406 to a distal opening 431 of distalmost face 428. First portion 413 may include an elastic material 440 surrounding and/or defining working channel 426 from the proximal end of shaft 406 to distalmost face 428 of distal tip 410.

Remaining portion 415 (e.g., a non-elastic portion) of shaft 406 may comprise a rigid, semi-rigid, or flexible but not elastic material. The side of shaft 406 that comprises remaining portion 415 may include an imaging device 430 and one or more light sources 432 at distal tip 410. The part of shaft 406 that includes or is adjacent to remaining portion 415 may include wires or cables (e.g., wires associated with imaging device 430 and light source(s) 432) and articulation members (e.g., articulation links 411 and cables) for articulating steering section 408. As better shown in FIGS. 4A and 4C, working channel 426 may be offset from articulation links 411. That is, working channel 426 does not extend through articulation links 411 in this example.

Working channel 426 may expand from the contracted state illustrated in FIGS. 4A and 4B to the expanded state illustrated in FIGS. 4C and 4D by stretching of elastic material 440 (e.g., due to medical instruments therethrough of larger size than working channel 426 in the contracted state). For example, working channel 426 when in a neutral position without medical instruments therein may be biased to the contracted state. As shown in FIGS. 4A, in the contracted state, working channel 426 may have a first diameter 1D. As shown in FIG. 4C, in the expanded state (e.g., accommodating one or more medical instruments), working channel 426 may have a second diameter 2D greater than first diameter 1D. Working channel 426 may expand to second diameter 2D due to, e.g., the insertion of a medical instrument (e.g., a laser fiber 442 or other instrument such as a basket device, etc.) having a diameter greater than 1D within working channel 426 and/or a force applied to a wall of working channel 426 by a medical instrument. In some examples, working channel 426 may expand uniformly. In some examples, working channel 426 may be expandable up to a diameter of approximately 3.6 French.

Shaft 406 may have a non-uniform outer diameter when working channel 426 is in the contracted state or in the expanded state. For example, in the contracted state, shaft 406 may have a first outer diameter 1D' at distal tip 410, a second outer diameter 2D' at an intermediate section 417, and a third outer diameter 3D' at remaining sections of shaft 406 proximal to intermediate section 417 (FIG. 4A). First outer diameter 1D' may be greater than second and third outer diameters 2D', 3D', and second outer diameter 2D' may be greater than third outer diameter 3D'. For example, in the expanded state, shaft 406 may have a first outer diameter 1D" at distal tip 410, a second outer diameter 2D" at intermediate section 417, and a third outer diameter 3D" at the remaining sections of shaft 406 proximal to intermediate section 417 (FIG. 4C). In the expanded state, first outer diameter 1D" may be greater than second and third outer diameters 2D", 3D", and second outer diameter 2D" may be greater than third outer diameter 3D".

An exemplary method of using shaft 406 in combination with handle 104 to treat kidney stones will now be described. Shaft 406 may be navigated through tortuous body anatomy of the urinary tract to a target site that includes kidney stones. Once shaft 406 is positioned proximate or at the target site, laser fiber 442 may be extended through port 116 of handle 104 and into working channel 426. Working channel 226 may expand due to a diameter of laser fiber 442 being greater than first diameter 1D of working channel 426. Once laser fiber 442 is positioned proximate or at the target site, laser fiber 442 may be used to fragment the kidney stones. Laser fiber 442 may then be removed from working channel 426 resulting in working channel 426 retracting back to the contracted state. Fluid may be delivered and/or suction may be applied to the target site through working channel 426 to clear out residual debris.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

1. A medical device comprising:
a shaft comprising:
a working channel extending from a proximal end of the shaft to an opening at a distal end of the shaft;
an imaging device at the distal end of the shaft; and
a plurality of optical fibers surrounding the working channel.

2. The medical device of claim 1, wherein the plurality of optical fibers completely surrounds the working channel.

3. The medical device of claim 1, wherein the plurality of optical fibers is arranged in a ring.

4. The medical device of claim 1, wherein the plurality of optical fibers defines walls of the working channel.

5. The medical device of claim 1, wherein the working channel has a diameter of approximately 3.6 French.

6. The medical device of claim 1, wherein the plurality of optical fibers surrounds the imaging device.

7. The medical device of claim 1, wherein the plurality of optical fibers is encapsulated by a material.

8. The medical device of claim 1, wherein the plurality of optical fibers comprises a coating.

9. The medical device of claim 1, wherein the plurality of optical fibers comprises epoxy resin adhesive or a UV-curable adhesive.

10. The medical device of claim 1, wherein each optical fiber of the plurality of optical fibers has an outer diameter of approximately 250 μm or less.

11. The medical device of claim 1, wherein the imaging device comprises a CMOS sensor.

12. The medical device of claim 1, wherein the shaft further comprises at least one irrigation channel or suction channel.

13. The medical device of claim 1, wherein the shaft has a non-circular cross-sectional shape.

14. The medical device of claim 1, wherein the shaft has an outer diameter less than approximately 9 French.

15. The medical device of claim 1, further comprising a handle coupled to the shaft, wherein the handle includes a port in communication with the working channel.

16. A medical device comprising:
a handle; and
a shaft extending distally from the handle, the shaft comprising:
a working channel extending from a proximal end of the shaft to an opening at a distal end of the shaft;
an imaging device at the distal end of the shaft; and
a plurality of optical fibers;
wherein the plurality of optical fibers defines walls of the working channel.

17. The medical device of claim 16, wherein an outer diameter of each optical fiber of the plurality of optical fibers is approximately 200 μm or less.

18. The medical device of claim 17, wherein the plurality of optical fibers is arranged in a ring.

19. A medical device comprising:
a shaft comprising:
a working channel extending from a proximal end of the shaft to an opening at a distal end of the shaft;
an imaging device at the distal end of the shaft; and
a light source at the distal end of the shaft;
wherein at least a portion of the shaft adjacent to the working channel from the proximal end of the shaft to the distal end of the shaft comprises an elastic material, the working channel being configured to expand by stretching of the elastic material.

20. The medical device of claim 19, wherein the shaft has a non-uniform outer diameter when the working channel is in a contracted state and when the working channel is in an expanded state.
